(19) United States
(12) Patent Application Publication
(10) Pub. No.: US 2026/0235571 A1
(43) Pub. Date: Aug. 13, 2026

(54) IMPROVED ALKALINITY MEASUREMENT

(71) Applicant: I-WATER, Stekene (BE)

(72) Inventors: Pieter VERMEIR, Stekene (BE); Thomas APERS, Stekene (BE)

(73) Assignee: I-WATER, Stekene (BE)

(21) Appl. No.: 19/156,830

(22) PCT Filed: Feb. 9, 2024
(86) PCT No.: PCT/IB2024/051225

§ 371 Date: Aug. 14, 2025

(30) Foreign Application Priority Data
Feb. 17, 2023 (BE) BE2023/5117
Feb. 17, 2023 (BE) BE2023/5118
Feb. 17, 2023 (BE) BE2023/5119

(51) Int. Cl. G01N 33/18; C02F 1/00; C02F 1/66; C02F 103/42; E04H 4/12; G01N 1/14

(52) U.S. Cl. CPC G01N 33/18; C02F 1/008; C02F 1/66; E04H 4/12; G01N 1/14; C02F 2103/42; C02F 2209/02; C02F 2209/06; C02F 2209/07

(57) ABSTRACT

The present invention provides, amongst other things, a device for measuring alkalinity of water, preferably water from a swimming pool, comprising: a first pump for automatically pumping a measuring liquid comprising a known dilution of an acid corresponding to a known acidity; a second pump for automatically pumping a sample of the water; a chamber connected to said first and second pump, for receiving a ready-to-measure solution comprising said sample and said measuring liquid according to a known mixing ratio, the chamber comprising a pH sensor for measuring acidity; a control unit connected to said pH sensor; wherein said control unit is configured to, based on a measurement of acidity of said ready-to-measure solution by means of said pH sensor and based on bijective relationship between acidity of the ready-to-measure solution and alkalinity of the water which is valid in a working area having said known acidity and said measurement of acidity, measure said alkalinity.

### TECHNICAL FIELD

The invention relates to a device and method for measuring alkalinity of water, a related system and a swimming pool, and the use of a pumping device for measuring alkalinity.

### STATE OF THE ART

Measuring water alkalinity is an important consideration in many applications, including monitoring pool water quality.

Existing methods and devices for measuring alkalinity are often too complex and/or require expensive instrumentation, requiring a set of various sensors. In some cases, measurement is also slow. Also, some form of integration with the measurement of other parameters which are important for water quality is often missing, and/or some form of correction based on the measurements. In addition, for many applications, including swimming pool applications, it is important that the substances and materials involved are non-toxic and that a general safe character can be guaranteed.

FR2935371A1, IN201911044176A, WO2017223365A1 and FR3005742A1 disclose related concepts but require a complex implementation and/or are not sufficiently accurate, which is disadvantageous.

There is a need for methods and devices for the cost-effective and reliable measurement of alkalinity. In doing so, the invention aims to solve the above-mentioned problems.

### SUMMARY OF THE INVENTION

The invention provides a device and method for measuring alkalinity of water, a system, a swimming pool, a use of a pumping device for measuring alkalinity; a method for correcting alkalinity of water, a use of a combination of liquids for automatic and liquid-based correction of alkalinity of a swimming pool, a kit for correcting alkalinity of a swimming pool; a method and device for automatically regulating quality of water, and a related swimming pool.

The various aspects are complementary and are mutually non-restrictive. Some aspects appear in the claims, other aspects also appear in the claims and are additionally referred to in this document as “item” or “clause”. This terminology is not restrictive in this regard, and the terms “item” and “clause” all serve to designate aspects and preferred embodiments of the invention which, like all claims, are patentable subject matter. The presence of a feature in one of the aspects is thereby non-limiting for the invention in its other aspects. A feature that is present in one aspect but not in one or more of the other aspects can thus be considered non-essential for at least all aspects in which the feature does not appear. This consideration further applies to all features, in each of the aspects.

In one aspect according to the claims, the invention provides a device for measuring alkalinity of water, preferably water from a swimming pool, comprising:
a first pump for automatically pumping a measuring liquid comprising a known dilution of an acid corresponding to a known acidity;
a second pump for automatically pumping a sample of the water;
a chamber connected to said first and second pumps, for receiving a ready-to-measure solution comprising said sample and said measuring liquid according to a known mixing ratio, the chamber comprising a pH sensor for measuring acidity;
a control unit connected to said pH sensor;
wherein said control unit is configured to, based on a measurement of acidity of said ready-to-measure solution by means of said pH sensor and based on a bijective relationship between acidity of the ready-to-measure solution and alkalinity of the water which is valid in a working area having said known acidity and said measurement of acidity, measure said alkalinity.

Such a device provides an answer to problems from the state of the art, see, for example, WO2017223365A1. There, a whole row of electrodes is required, which can make it expensive and cumbersome to measure alkalinity. Moreover, the method according to WO2017223365A1 can be slow, as it requires working towards an equivalence point, where the power consumed during titration is a measure of alkalinity. The present invention provides an alternative by requiring only a pH sensor for measuring alkalinity, so that, for example, a pair of electrodes may suffice. Also, the solution, which is obtained by mixing measuring liquid and sample, is “ready-to-measure”, so that it is not required to work towards an equivalence point when measuring, which may improve the speed of measurement, and/or save electrical energy, and/or avoid the oxidation which is inherent in titration.

In an aspect according to the claims, the invention provides a system comprising the device, a source of measuring liquid and a set of connections for measuring alkalinity according to a closed circuit with an inlet connected to said second pump, and an outlet connected to said chamber.

This system has advantages similar to those of the corresponding device.

In one aspect, the invention provides a swimming pool comprising the system comprising said inlet and outlet and a pool tub comprising said water connected to said system via said inlet and outlet.

This swimming pool has advantages similar to those of the corresponding system and device.

In an aspect, the invention provides a method for measuring alkalinity of water, preferably water from a swimming pool, comprising the steps:
having a measuring liquid mixed with a sample of water according to a known mixing ratio, the measuring liquid comprising a known dilution of an acid corresponding to a known acidity, to obtain a ready-to-measure solution;
performing, with regard to said ready-to-measure solution, a measurement of acidity by means of a pH sensor;
measuring said alkalinity of water, based on said measurement of acidity and based on a bijective relationship between acidity and alkalinity which is valid in a working area having said known acidity and said measured acidity.

Such a method is advantageous because it can avoid the disadvantages of alkalinity measurement according to the prior art. In WO2017223365A1, alkalinity measurement is related to the generation of hydronium using the titrant-generating electrode, thereby changing the acidity. The generation is thereby aimed at reaching a predetermined end point of electrochemical titration, i.e. achieving titration to equivalence point. Thereby, the amount of power of current consumed, or the amount of hydronium ions generated, is indicative of alkalinity. This measurement principle is thus based on a correlation, where generation and detection are correlated. In the alkalinity measurement according to the present invention, there is no such complex correlation, and the starting point is the same amount of acid, as a result of which the mixture of measuring liquid and sample is immediately ready-to-measure. This makes the method simpler and more cost-effective, may lead to faster and/or more energy-efficient measurement, and may also lead to a more robust implementation. Indeed, by avoiding electrochemical titration, the known usage problems of electrodes (e.g., due to transition resistance) are avoided. The consumption of measuring liquid can also easily be predicted, as a dosed quantity of measuring liquid is administered each time. In exemplary embodiments, this is always the same quantity.

In one aspect, the invention provides a use of a pumping device for measuring alkalinity, by pumping a sample of water and a measuring liquid according to a known mixing ratio by means of devices, belonging to the pumping device, for realising a mechanically coupled volumetric ratio.

This usage can provide an advantageous solution to the problem of realising a ready-to-measure solution according to a known mixing ratio. Rather than working with two pumps whose flow rate can be independently controlled (two degrees of freedom), it may be advantageous to essentially control the mutual ratio, by said mechanically coupled volumetric ratio (one degree of freedom). Dosage is also relevant, but may have less influence on the accuracy of alkalinity measurement.

In an aspect according to the claims and according to the clauses herein, the invention provides a method for correcting alkalinity of water, preferably water from a swimming pool, comprising the steps:
automatically pumping a sample of said water and a measuring liquid comprising a known dilution of an acid corresponding to a known acidity, according to a known mixing ratio, to obtain a ready-to-measure solution;
measuring an acidity of said ready-to-measure solution by means of a pH sensor;
measuring, based on said acidity, said alkalinity of the water;
performing, based on said measurement, said correction with regard to the alkalinity of the water, by an automated and dosed addition of an alkalinity-increasing liquid by means of an alka-plus pump.

An advantage of such a method relates to the fully liquid-based measurement and correction of alkalinity, with only a pH sensor required as a minimum. This can offer easy maintenance, requiring only two liquids to be replenished, and where possible wear on sensors can be limited to a small number of sensors, in embodiments only the pH sensor. Such a method of correction is not described or suggested by the state of the art.

In an aspect according to the claims and according to the clauses herein, the invention provides a use of a combination of a measuring liquid comprising a known dilution of an acid corresponding to a known acidity comprising adipic acid and an alkalinity-increasing liquid comprising a carbonate or bicarbonate for automatic and liquid-based correction of alkalinity of a swimming pool.

An advantage of such use relates to the fully liquid-based measurement and correction of alkalinity. This can offer simple maintenance, requiring only two liquids to be replenished, which can be cost-saving due to the similar nature of maintenance. Here, an operator can match supplies so that both supplies are replenished with just one intervention, which is even more cost-effective.

In an aspect according to the claims and according to the clauses herein, the invention provides a kit for correcting alkalinity of a swimming pool, comprising:
a measuring liquid comprising a known dilution of an acid corresponding to a known acidity comprising adipic acid; and an alkalinity-increasing liquid comprising a carbonate or bicarbonate;
wherein said alkalinity-increasing liquid comprises carbonate, CO₃²⁻, and/or bicarbonate, HCO₃⁻, and wherein said alkalinity-increasing liquid comprises said carbonate or bicarbonate at a concentration higher than 0.2 M.

An advantage of such a kit relates to fully liquid-based measurement and correction of alkalinity. This can offer easy maintenance, with two liquids being replenished simultaneously, with supplies mutually matched according to a kit, so that only one delivery is needed.

In an aspect according to the claims and according to the items herein, the invention provides a method for automatically regulating water quality, preferably water from a swimming pool, comprising the steps:
actively measuring alkalinity of water with a first sensor, during a first state;
switching, from the first to a second state;
measuring pH of the water with the same first sensor, during the second state;
calculating, based on said measurement of alkalinity and pH, an index for water balance, preferably a Langelier Saturation Index, LSI; determining whether the calculated index meets a predetermined criterion regarding said quality;
if said criterion is not met, generating an instruction for correcting said quality;
wherein said first sensor is a pH sensor;
wherein said first and second states are at least partially non-overlapping in time, preferably non-overlapping in time. In preferred embodiments, said active measurement relates to the automated and dosed addition of a measuring liquid for obtaining a ready-to-measure solution. In alternative embodiments, said active measurement relates to an alkalinity measurement with ranges of equivalence point as described, for example, in WO2017223365A1, wherein the measurement of alkalinity preferably takes place with a second sensor which is at least partially different from the pH sensor used for the measurement of pH.

An advantage of such a method is its far-reaching automation, wherein said switching makes it possible to measure both pH and alkalinity, requiring only a pH sensor as a minimum. This is made possible by the active form of measurement, which, for example, is related to mixing a measuring liquid and a sample, but may also be related to an alkalinity measurement with ranges of equivalence point as described, for example, in WO2017223365A1. However, in contrast to WO2017223365A1, a method related to mixing a measuring liquid and a sample offers both alkalinity measurement and pH measurement using the same single pH sensor, consisting, for example, of only two electrodes, which is advantageous. This can offer easy maintenance, where possible wear on sensors can be limited to a small number of sensors, in embodiments only the pH sensor. Such a method for automatically regulating water quality is not described or suggested by the state of the art.

In an aspect according to the claims and according to the items herein, the invention provides a device for automatically regulating water quality, comprising:
a first sensor;
means for performing an active measurement;
a control unit connected to said first sensor;
wherein said control unit is configured for:
measuring, by means of said first sensor, pH of the water, during a first state;
actively measuring, by means of a second sensor, which is preferably equal to the first sensor, and said means for performing said active measurement, said alkalinity of the water, during a second state;
calculating, based on said measurement of pH and alkalinity, an index for water balance, preferably a Langelier Saturation Index, LSI;
determining whether the calculated index meets a predetermined criterion regarding said quality;
if said criterion is not met, generating an instruction for improving said quality;
wherein said first sensor is a pH sensor;
wherein said first and second states are at least partially non-overlapping in time, preferably non-overlapping in time;
wherein said means for performing the active measurement are preferably related to the automated and dosed addition of a measuring liquid for obtaining a ready-to-measure solution.

The advantages of such a device are similar to those of the corresponding method.

In an aspect according to the claims and according to the items herein, the invention provides a swimming pool comprising the device according to the items herein comprising an inlet and outlet and a pool tub comprising said water connected to said device by means of said inlet and outlet.

This swimming pool has advantages similar to those of the corresponding device.

Embodiments according to the dependent claims and their respective potential advantages are explained in the detailed description.

### DESCRIPTION OF THE FIGURES

FIG. 1 shows a schematic exemplary embodiment of a device according to the invention.

FIGS. 2A-2B show exemplary embodiments of devices according to the invention. Thereby, FIG. 2A shows an exemplary embodiment without incoming-flow pump, and FIG. 2B shows an exemplary embodiment with incoming-flow pump.

FIGS. 3A-3B show exemplary embodiments of devices according to the invention switching between a first and second state. Thereby, FIG. 3A shows an exemplary embodiment with valve, and FIG. 2B shows an exemplary embodiment with incoming-flow pump.

FIG. 4 shows a schematic exemplary embodiment of a device according to the invention with an alka-plus pump.

FIG. 5 shows an exemplary embodiment of a device according to the invention with both an alka-plus pump and a pH-min pump.

FIG. 6 shows an example of a bijective relationship according to the invention, wherein alkalinity of water is plotted as a function of acidity of the ready-to-measure solution.

### DETAILED DESCRIPTION

Unless otherwise defined, all terms used in the description of the invention, including technical and scientific terms, have the meaning as generally understood by those skilled in the technical field of the invention. For a better understanding of the description of the invention, the following terms are explicitly explained. “A”, “the” and “it” refer to both singular and plural in this document unless the context clearly assumes otherwise. For example, “a segment” means one or more than one segment. Citations of numeric intervals by endpoints include all integers, fractions and/or real numbers between the endpoints, these endpoints included.

In this document, the term “water” refers to an aqueous solution most of which is H₂O. Throughout this document, “water” can be replaced by the term “aqueous solution” without this leading to a reassessment of the scope of the invention or the claims. The term “water” is thus an umbrella term for all aqueous solutions that would in everyday practice be referred to by the term water. In preferred embodiments, the aqueous solution consists of at least 80% H₂O, more preferably at least 90% H₂O, most preferably at least 98% H₂O. The water may be related to any form of fresh water, salt water, sea water, surface water, groundwater, drinking water, process water. In embodiments, the water relates to water from a swimming pool, pond, swimming pond or breeding pond.

In this document, “alkalinity” is a measure of the acid-buffering capacity of water. This can relate to both a qualitative and quantitative measure. Here, alkalinity relates to the ability of the water, i.e. the aqueous solution, to neutralise acids. Preferably, it relates to the stoichiometric sum of the bases in the aqueous solution, such as hydroxide ions (OH⁻), carbonate ions (CO₃⁻) and hydrogen carbonate ions (HCO₃⁻). Alkalinity is a measure the buffering capacity of water, i.e., its ability to prevent pH fluctuations. If alkalinity is too low, the pH will fluctuate strongly upon addition of an acid or a base. If alkalinity is too high, pH will be difficult to correct.

In this document, the term “bijective relationship” is preferably related to a calibration curve, more preferably a calibration line, which determines the relationship between alkalinity and acidity.

In this document, “acidity” is an alternative term to “pH”, where the former term may be substituted for the latter, whereas the latter refers to both acidity and a measurement of acidity. A “measurement of acidity”, or a “measurement of pH”, thereby corresponds to a pH value.

In this document, the terms “volume” and “flow rate” are interchangeable. Indeed, in embodiments, the respective known volumes of said sample and said dose are functions of respective known flow rates. Thereby, the volume then corresponds to the volume that, for the known flow rate, can be pumped by the respective pump during a certain time window, e.g. a certain cycle (with or without time measurement), or a certain measured period.

In this document, the term “molar concentration (or molarity)” refers to the degree of dilution of a substance. It is expressed in “M” or “molar”, corresponding to mol per litre (mol/l).

In embodiments, said known mixing ratio is further related to the pumping of a known volume of said measuring liquid, with said first pump, and/or a known volume of a sample of said water, with said second pump. This may have the advantage that the mixing ratio is more precisely known, wherein it is calculated both based on the mechanically coupled volumetric ratio between the flow rates of the pumps themselves, and based on one, or both, of the respective flow rates of the pumps.

In alternative embodiments, said known mixing ratio does not relate to a mechanically coupled volumetric ratio between the flow rate of the first pump and the flow rate of the second pump, but instead it relates to the pumping of a known volume of said measuring liquid, with said first pump, and a known volume of a sample of said water, with said second pump.

In embodiments, measuring alkalinity is related to water quality analysis.

In embodiments, the pH sensor comprises a set of a first and second electrode. These may include both solid-state electrodes and glass electrodes. Further, the invention is not limited thereto, and may use any means suitable for pH metrics, including holographic pH sensors with colorimetric analysis, and including forms of pH metrics without the use of electrodes.

In preferred embodiments, the acid present in the measuring liquid is related to adipic acid. In preferred embodiments, the concentration of adipic acid present in the measuring liquid is between 0.01 g and 10 g per litre, more preferably between 0.1 g and 1 g per litre, most preferably 0.2 or 0.3 or 0.4 or 0.5 or 0.6 or 0.7 or 0.8 or 0.9 g per litre. In preferred embodiments, the mixing ratio of measuring liquid and sample of water is between 1:4 and 4:1, more preferably between 1:3 and 3:1, most preferably 1:2 or 2:3 or 3:4 or 4:5 or 1:1 or 5:4 or 4:3 or 3:2 or 2:1. In preferred embodiments, the mixing ratio is a function of the mechanically coupled volumetric ratio between the flow rate of the measuring liquid and the flow rate of the sample. This ratio depends on the precise characteristics of the pumps and/or the pumping device, and can be any real number. In preferred embodiments, this number is determined during calibration and taken into account when calculating a measured value of alkalinity as a function of acidity measurement.

In WO2017223365A1, a pH-sensing electrode and the titrant-generating electrode are disclosed for determining alkalinity. Thus, contrary to the present invention, a titrant-generating electrode is required there, whereas the present invention starts from a measuring liquid. Importantly, the operation of the measurement also differs. In WO2017223365A1, the alkalinity measurement relates to the generation of hydronium using the titrant-generating electrode, thereby changing the acidity. The generation is thereby aimed at reaching a predetermined end point of electrochemical titration, i.e. achieving titration to equivalence point. Thereby, the amount of power or current consumed, or the amount of hydronium ions generated, is indicative of alkalinity. This measurement principle is thus based on a correlation, where generation and detection are correlated. In the alkalinity measurement according to the present invention, there is no such complex correlation, and the starting point is the same amount of acid, as a result of which the mixture of measuring liquid and sample is immediately ready-to-measure. This makes the measurement simpler and more cost-effective, may lead to faster and/or more energy-efficient measurement, and may also lead to a more robust implementation. Indeed, by avoiding electrochemical titration, the known usage problems of electrodes (e.g., due to transition resistance) are avoided. The consumption of measuring liquid can also easily be predicted, as a dosed quantity of measuring liquid is administered each time. In exemplary embodiments, this is always the same quantity.

In exemplary embodiments, the cycle of switching, between the first and second states, in the domain of swimming pools, consists of a short measurement of alkalinity, alternating with a long period (e.g. 2-4 hours) of continuous measurement of pH. In other exemplary embodiments, there is rapid switching between measurement of alkalinity, switching, for example, every three or five or ten minutes between a measurement of alkalinity, for a period of the given duration, and a measurement of pH, for a period of the same duration.

The prior art discloses the use of two electrode sets for alkalinity measurement, see, for example, WO2017223365A1. In contrast to the present invention, alkalinity measurement there relies solely on coulometric titration. There, the first electrode set monitors the pH and the second electrode set provides the coulometric titration. In the example of WO2017223365A1, the second set relates to a hydronium-generating electrode and a hydroxide ion-generating electrode, where the role can be switched (by reversing the current sense). The pH-detecting electrode and the hydronium-(or hydroxide-) producing electrode(s) can be called working electrodes. Each of the electrode sets also contains at least one reference electrode and at least one first counter electrode, wherein the sets may share the same reference and counter electrodes. Thus, despite this sharing of an electrode, according to WO2017223365A1, such an arrangement requires a whole row of electrodes, making the implementation thereof expensive and cumbersome. Moreover, it can be slow because it requires working towards an equivalence point, where the power consumed during titration is a measure of alkalinity. The present invention offers an alternative by working only with a pH sensor, so that, for example, a pair of electrodes can suffice. Also, the solution which is obtained by mixing measuring liquid and sample is “ready-to-measure”, so it is not required to work towards an equivalence point when measuring.

In embodiments, said known mixing ratio relates to a mechanically coupled volumetric ratio between the flow rate of the first pump 10 and the flow rate of the second pump 70. In embodiments having a common pump body, it may be, for example, a diaphragm pump or peristaltic pump; however, it may be by any other means. It may also involve, for example, a fixed transmission ratio, for example with pulleys and/or gears, where the actuation of one pump, for example, is done by transmission of the rotation of the shaft of the other pump. Such mechanical coupling may be advantageous because it exhibits predictable behaviour, and does not require separate monitoring of mixing ratio. Thereby, it can be further advantageous that it is the ratio that is coupled, rather than individual volumes (or, equivalently, flow rates). Indeed, when holding or controlling individual volumes, there are two degrees of freedom, whereas the invention allows only taking into account the mixing ratio, i.e. one degree of freedom, without being too sensitive to the precise volume of the ready-to-measure solution. Moreover, if this mixing ratio is realised by mechanical coupling, it can advantageously be constant over time, or at least exhibit a predictable behaviour, where a dependence on, for example, temperature can be overcome by software (e.g., in the calculations made by the control unit). Such predictability allows the mixing ratio of the first and second pumps to be determined at calibration, and then to be taken into account when further calibrating the device. For example, the mixing ratio obtained in this way can be used as input when calibrating the control unit. In other examples, further calibration of the control unit can even be avoided by adjusting the dilution of the acid present in the measuring liquid. The latter can even be done completely manually, which is convenient and fast, and thus advantageous.

In embodiments, said transmission of rotation relates to:
pulleys, said transmission ratio being related to a ratio of respective diameters of said pulleys; and/or
gears, said transmission ratio being related to a ratio of respective number of teeth of said gears.

In embodiments with pulleys, the transmission preferably relates to at least one drive belt. In embodiments, the drive belt relates to a smooth belt or V-belt, and the pulleys on their outer surface preferably comprise a V-shaped cross-section for guiding the belt. In embodiments, the drive belt, either alternatively or additionally, relates to a toothed belt, and the pulleys on their outer surface preferably comprise an outer toothing adapted for slip-free connection between the outer surface and the toothed belt.

In embodiments, transmission is related to both gears and pulleys.

In embodiments, the device comprises an outlet connected to said chamber, configured for draining the ready-to-measure solution to the water according to a closed circuit.

In embodiments, the device comprises a mixing coil provided between said first pump and second pump and said chamber for mixing said measuring liquid and said sample of water to said ready-to-measure solution.

In embodiments, the device comprises a temperature sensor for measuring temperature of said ready-to-measure solution; wherein said control unit is further configured to measure the temperature of said ready-to-measure solution by means of the temperature sensor and based thereon to convert said measurement of acidity for obtaining an equivalent pH, wherein said bijective relationship between acidity and alkalinity is applied to said equivalent pH for said measurement of alkalinity.

In embodiments, the device comprises means for switching between a first state and a second state, wherein said first state corresponds to measuring alkalinity of the water by means of said pH sensor; and wherein said second state corresponds to measuring acidity of the water by means of the same pH sensor.

In embodiments, said means of switching are related to a valve, preferably a three-way valve; wherein said first state is associated with actuation of said first pump and second pump and with maintaining, by means of said valve, an incoming flow of water without passage through the chamber, for said measurement of alkalinity by means of the pH sensor; and wherein said second state is associated with non-actuation of said first pump and second pump and with guiding, by means of said valve, the incoming flow of water through the chamber, for said measurement of acidity by means of the pH sensor.

In embodiments, said means of switching are related to an incoming-flow pump;
wherein said first state is associated with actuation of said first pump and second pump and with non-actuation of the incoming-flow pump, for said measurement of alkalinity by means of the pH sensor; and
wherein said second state is associated with non-actuation of said first pump and second pump and with actuation of the incoming-flow pump for guiding the incoming flow of water through the chamber, for said measurement of acidity through the pH sensor.

In embodiments, the device comprises an alka-plus pump, i.e., a dosing pump for automatically adding, based on said measurement of alkalinity of the water, an alkalinity-increasing liquid to the water. In embodiments, the alka-plus pump is used for increasing alkalinity. In embodiments, the alka-plus pump is, alternatively or additionally, used for increasing the acidity of the water, wherein the alkalinity-increasing liquid is related to a base such as, for example, bicarbonate. Thereby, the influence of alkalinity-increasing liquid on both alkalinity and acidity is preferably taken into account, preferably via index-based conversion.

In embodiments, the control unit is configured to correct at least one of alkalinity and acidity of the water, preferably both alkalinity and acidity, by a choice of adding the alkalinity-increasing liquid and/or the acidity-reducing liquid. For example, this relates to correcting alkalinity using the acidity-reducing liquid. In another example, this relates to correcting acidity using the alkalinity-increasing liquid. In yet another example, this relates to correcting acidity using both the acidity-reducing liquid and the alkalinity-increasing liquid. In yet another example, this relates to correcting alkalinity using both the acidity-reducing liquid and the alkalinity-increasing liquid. This may have the advantage of enabling a more accurate correction.

In embodiments, the device comprises a pH-min pump for automatically adding, based on one of said measurements of acidity, an acidity-reducing liquid to the water, said measurement of acidity being related to:
said measurement of acidity of said ready-to-measure solution; and/or
if available, said measurement of acidity of the water.

In embodiments in which only the acidity of the ready-to-measure solution and not the acidity of the water itself is measured, the addition is made on the basis of the measurement of alkalinity, which is determined on the basis of at least the measurement of acidity of the ready-to-measure solution. In embodiments, the pH-min pump is used to reduce the acidity of the water. In embodiments, the pH-min pump is, alternatively or additionally, used to reduce alkalinity. Thereby, the influence of acidity-reducing liquid on both alkalinity and acidity is preferably taken into account, preferably via index-based conversion.

In embodiments, the device comprises a salt-plus pump for automatically adding, based on said measurement of alkalinity and/or based on one of said measurements of acidity, a salinity-increasing liquid to the water. In embodiments in which only the acidity of the ready-to-measure solution and not the acidity of the water itself is measured, the addition is made on the basis of the measurement of alkalinity, which is determined on the basis of at least the measurement of acidity of the ready-to-measure solution.

In embodiments, the device comprises said alka-plus pump and said pH-min pump; wherein said control unit is further configured to, based on repeatedly performing said measurement of alkalinity of the water and said acidity of the water, perform a correction with respect to the water, wherein the correction includes both the possibility of adding said alkalinity-increasing liquid by means of said alka-plus pump and the possibility of adding said acidity-reducing liquid by means of said pH-min pump and, preferably, further includes the possibility of adding said salinity-increasing liquid by means of said salt-plus pump.

In embodiments, said adding of alkalinity-increasing liquid occurs in priority to at least said adding of acidity-reducing liquid in order to make the correction gradual.

In embodiments, said correction includes the possibility of adding said salinity-increasing liquid by means of said salt-plus pump, and wherein said adding of salinity-increasing liquid occurs in priority to at least said adding of acidity-reducing liquid in order to make the correction gradual.

In embodiments, said mixing is done by means of a first pump for said measuring liquid and a second pump for said sample, wherein the first and second pumps are mechanically coupled according to a volumetric ratio between the flow rate of the first pump and the flow rate of the second pump.

In embodiments, said steps are preceded by the steps of:
calibrating the pH sensor with at least two pH calibration solutions corresponding to a pH-related bijective calibration curve that relates pH to a value measured by said pH sensor, preferably the voltage between a pair of electrodes belonging to the pH sensor;
calibrating with regard to alkalinity with at least two alkalinity calibration solutions corresponding to the above-mentioned bijective relationship between acidity of the ready-to-measure solution and alkalinity of the water.

In embodiments, said alkalinity-increasing liquid comprises carbonate, CO₃²⁻, and/or bicarbonate, HCO₃⁻. In preferred embodiments, said alkalinity-increasing liquid thereby comprises a buffered pH between pH 5 and pH 9, preferably between pH 6 and pH 8. More preferably, the alkalinity-increasing liquid is ph neutral, or, equivalently, has a buffered pH of about pH 7. This relates, for example, to a solution of a carbonate and/or bicarbonate which additionally comprises a buffer, in order to achieve a buffered pH for the solution. This involves buffering the pH as known to the skilled worker, a mechanism known for example in buffering by weak acids, such as acetic acid. An advantage of such an alkalinity-increasing liquid with buffered pH is that it can be added to water (e.g. the water of a swimming pool) without causing a pH fluctuation. This can allow a targeted and faster adjustment of the water balance, with faster and more direct setting of a desired equilibrium point.

In embodiments, said alkalinity-increasing liquid includes said carbonate or bicarbonate at a concentration higher than 0.2 M.

In embodiments, said alkalinity-increasing liquid comprises potassium, preferably potassium derived from dissolving potassium carbonate, K₂CO₃, and/or potassium bicarbonate, KHCO₃, in water, for obtaining the alkalinity-increasing liquid.

In embodiments, said alkalinity-increasing liquid comprises sodium, preferably sodium derived from dissolving sodium carbonate, Na₂CO₃, and/or sodium bicarbonate, NaHCO₃, in water, for obtaining the alkalinity-increasing liquid.

In embodiments, said automated pumping of respective samples of water and of measuring liquid and said measuring of acidity of said ready-to-measure solution and alkalinity of the water is repeated periodically, wherein said correction is performed by adding alkalinity-increasing liquid when a difference between a target value for alkalinity and the measured alkalinity exceeds a certain threshold.

In embodiments, said dosage for correcting alkalinity of the water by adding alkalinity-increasing liquid corresponds to a corrected value not exceeding said target value, preferably lower than said target value.

In embodiments, said measurement of said acidity is done by means of said pH sensor on the basis of a bijective relationship between acidity of the ready-to-measure solution and alkalinity of the water which is valid in a working area having said known acidity and said measurement of acidity.

In embodiments, said diluted acid belonging to said measuring liquid relates to dicarboxylic acid, preferably adipic acid, C₆H₁₀O₄.

In embodiments, said dicarboxylic acid is adipic acid, wherein said dilution is preferably situated between 0.01 and 10 g/l, more preferably between 0.1 and 1 g/l, most preferably is 0.2 or 0.3 or 0.4 or 0.5 or 0.6 or 0.7 or 0.8 g/l.

In embodiments, said known mixing ratio is between 1:10 and 10:1, preferably between 1:5 and 5:1, more preferably between 1:4 and 4:1 or between 1:3 and 3:1 or between 1:2 and 2:1, most preferably between 2:3 and 3:2 or between 3:4 and 4:3.

In embodiments, the method further comprises the step:
measuring temperature of said ready-to-measure solution by means of a temperature sensor;
wherein said measurement of acidity of said ready-to-measure solution is converted based on said temperature measurement for obtaining an equivalent pH, wherein said bijective relationship between acidity and alkalinity is applied to said equivalent pH for said measurement of alkalinity.

In embodiments, the method further comprises the step:
switching between a first state and a second state, said first state corresponding to measuring alkalinity of the water by means of said pH sensor; and said second state corresponding to measuring acidity of the water by means of the same pH sensor;
wherein said correction of alkalinity is done on the basis of both said measurement of alkalinity of water and said measurement of acidity of water.

In embodiments, the method further comprises the step:
performing, based on both said measurement of alkalinity of the water and said measurement of acidity of the water, a correction with regard to the acidity of the water, by an automated and dosed addition of an acidity-reducing liquid by means of a pH-min pump.

In embodiments, said water is the water of a swimming pool.

In embodiments, said water balance index is the LSI.

In embodiments, said criterion relates to a warning interval, wherein if the calculated index is not within said interval, an instruction is generated warning the operator.

In embodiments, said criterion relates to a correction interval, wherein if the calculated index is not within said interval, an instruction for correction is generated, and wherein said method further comprises at least one of the following two steps:
if said instruction for correction includes increasing alkalinity, the automated and dosed addition of an alkalinity-increasing liquid to the water by means of an alka-plus pump; and/or
if said instruction for correction includes lowering acidity, the automated and dosed addition of an acidity-reducing liquid to the water by means of a pH-min pump. Preferably, the method includes both of the above two steps.

In embodiments, said measurement of said acidity is done by means of said pH sensor based on a bijective relationship between acidity of the ready-to-measure solution and alkalinity of the water which is valid in a working area having said known acidity and said measurement of acidity.

In embodiments, said active measurement relates to having a measuring liquid mixed with a sample of the water according to a known mixing ratio, the measuring liquid comprising a known dilution of an acid corresponding to a known acidity, for obtaining the ready-to-measure solution.

In embodiments, said known mixing ratio is between 1:10 and 10:1, preferably between 1:5 and 5:1, more preferably between 1:4 and 4:1 or between 1:3 and 3:1 or between 1:2 and 2:1, most preferably between 2:3 and 3:2 or between 3:4 and 4:3.

In embodiments, the method further comprises the step of:
measuring temperature of said ready-to-measure solution by means of a temperature sensor;
wherein said measurement of acidity of said ready-to-measure solution is converted based on said temperature measurement for obtaining an equivalent pH, wherein said bijective relationship between acidity and alkalinity is applied to said equivalent pH for said measurement of alkalinity.

In embodiments, said method further comprises the step of:
repeatedly switching between the first state and the second state;
wherein said steps are repeatedly completed based on repeated measurement of alkalinity of the water, during the first state, and of acidity of the water, during the second state.

In embodiments, said criterion relates to an interval for correction, wherein if the calculated index is not within said interval, an instruction for correction is generated, and wherein said method comprises the following step:
if said instruction for correction includes increasing salinity, the automated and dosed addition of a salinity-increasing liquid to the water by means of a salt-plus pump.

In embodiments, said addition of alkalinity-increasing liquid is done in priority to at least said addition of acidity-reducing liquid in order to make the correction gradual.

In embodiments, said method further comprises the following step:
if said instruction for correction includes increasing salinity, the automated and dosed addition of said salinity-increasing liquid to the water by means of said salt-plus pump;
wherein said addition of salinity-increasing liquid occurs in priority to at least said addition of acidity-reducing liquid in order to make the correction gradual.

In embodiments, said device regulates the water quality by means of a ready-to-measure solution, and said swimming pool includes a further pump realising an incoming flow through said device for draining the ready-to-measure solution to the pool tub.

In preferred embodiments, the diluted acid which is present in the measuring liquid relates to adipic acid or, equivalently, hexanoic acid. An advantage of this may be the ready availability of this acid, with a large global production annually, which is related, among other things, to the usefulness of this acid in certain nylon production processes. A further advantage is its non-toxic nature, as evidenced by the E number for adipic acid as a food additive, E355. The non-toxic nature is advantageous for general application, and is particularly advantageous for use for the water of a swimming pool.

In preferred embodiments, the Langelier saturation index (LSI) is used as an index of water quality or water balance. The LSI is thereby a measure of saturation to calcium carbonate (CaCO₃). An LSI value which is too low is associated with corrosion, whereas a value which is too high is associated with scale forming. Perfect saturation corresponds to 0.00 LSI, and an acceptable range of LSI is, for example, a value between −0.30 and +0.30 LSI. By keeping water quality within the acceptable range, both corrosion and scale forming can be prevented as much as possible.

The LSI is a function of a set of various parameters, for example five or six, thereby providing a holistic view on the water balance, with the influence of the various parameters accounted for in one single value. In this way, the LSI provides a pragmatic tool for monitoring water quality, as a predictor of corrosion or scale forming. This is superior to the common method in the state of the art, to monitor only certain intervals, where the pH must be, for example, between 7.2 and 7.8, preferably between 7.4 and 7.6, and/or where calcium must be kept in the range of 200-400 ppm, or total alkalinity must be kept between 80 and 120 ppm. Monitoring such intervals is a complex challenge, which can be disadvantageous. Moreover, monitoring intervals only gives an incomplete view, which can lead to poor results. Indeed, corrosion and/or scale forming is not determined by the intervals separately, but by the values in combination. Thus, it is possible that certain combinations of values are all in their respective intervals, but that corrosion or scale forming still occurs.

In embodiments, the LSI is a function of a set of two or more different parameters of: pH, water temperature (T) (expressed in ° C.), calcium hardness (CH) (expressed in ppm), carbonate alkalinity (CA), cyanuric acid/stabiliser (CYA) (expressed in ppm), total alkalinity (ALKA, expressed in ppm), total dissolved solids (TDS) (expressed in ppm), and electrical conductivity (EC) (expressed in ppm).

In embodiments with six parameters, the index, preferably the LSI, relates, for example, to pH, water temperature (T) (expressed in ° C.), calcium hardness (CH) (expressed in ppm), carbonate alkalinity (CA), cyanuric acid/stabiliser (CYA) (expressed in ppm), and total dissolved solids (TDS) (expressed in ppm).

In embodiments with five parameters, the index, preferably the LSI, relates, for example, to pH, water temperature (T) (expressed in ° C.), calcium hardness (CH) (expressed in ppm), total alkalinity (ALKA, expressed in ppm), and electrical conductivity (EC) (expressed in ppm). Here, total alkalinity (ALKA) relates to carbonate alkalinity (CA), cyanuric acid/stabiliser (CYA) and pH. Electrical conductivity (EC), in turn, relates to total dissolved solids (TDS). Conductivity measurement is done with an electronic sensor in micro/milli-Siemens per centimetre or ppm, preferably in ppm. Conductivity increases with increasing ion content, meaning that in most cases, there is a simple relationship with a TDS measurement, with, for example, a conversion factor CF of 1 ppm=CF-uS/cm, where CF is, for example, 2. Conductivity is temperature-sensitive and is typically standardised at 25° C.

In exemplary embodiments, the LSI relates to the following formula:

$$LSI = ( pH ) + ( - 0.00014554 * ( T in °C ) * ( T in °C ) + 0.02808929 * ( T in °C ) + 0.00408167 ) + ( 0.43475046 * ln ( Calcium hardness in ppm ) - 0.39514691 ) + ( 0.43475046 * ln ( ALKA in ppm ) + 0.00485309 ) - ( - 1. \times {10}^{- 8} * ( EC in ppm ) * ( EC in ppm ) + 0.000122 * ( EC in ppm ) - 12.08 )$$

In variants, the LSI is expressed in °iW, which is a scaled version of the LSI, according to the conversion:

$$∘ IW = 5 / 3 * LSI + 100.$$

The skilled worker thereby takes into account possible minor deviations and variants as to the shape of the LSI function, both in terms of coefficients and the precise nature of proportionality (linear vs non-linear, logarithm or other elementary function, etc). Embodiments with deviations and variants as to the shape of the LSI function are thereby in full covered by the invention.

In preferred embodiments, the water balance is regulated according to threshold values. In exemplary embodiments, this relates to one or more, preferably all, of the following values:
LSI
   Maximum: M1 (Scale forming)
   Warning limit high: W1
Ideal: 0
Warning limit low: w1
Minimum: m1 (corrosive)
   °iW
Maximum: M2 (Scale forming)
Warning limit high: W2
Ideal: 100
Warning limit low: w2
Minimum: m2 (corrosive)

Preferably, M1 and −m1 are in the range of 0.2-0.4, more preferably in the range of 0.25-0.35, and most preferably they are equal to 0.3. Preferably, M1=−m1.

Preferably, W1 and −w1 are in the range of 0.05−0.3, more preferably in the range of 0.10-0.20, and most preferably they are equal 0.15. Preferably, W1=−w1.

Preferably, M2 is in the range of 100-250, more preferably in the range of 120-180, and M2 is most preferably equal to 150. Preferably, m2 is in the range of 0-100, more preferably in the range of 25-75, and m2 is most preferably equal to 50. Preferably M2−100=100−m2.

Preferably, W2 is in the range of 100-150, more preferably in the range of 115-135, and W2 is most preferably equal to 125. Preferably, w2 is in the range of 50-100, more preferably in the range of 65-85, and w2 is most preferably equal to 75. Preferably, W2−100=100-w2.

In embodiments, monitoring the water balance, e.g. via the LSI, is related to performing a correction with respect to the water. In the present invention, this correction is preferably related to the addition of an alkalinity-increasing liquid. This contrasts with the approach in the prior art, as for example in IN201911044176A and FR3005742A1. Although, in IN201911044176A, monitoring of the LSI is described, corrections are made only with chlorine, algaecides and stabilisers. As a result, in methods according to IN201911044176A, it is not possible to directly overcome low alkalinity, which means that in many cases, the LSI cannot be brought back to a desired range. In contrast, in embodiments related to monitoring the water balance, the present invention provides the possibility of correcting alkalinity directly and automatically, using the alkalinity-increasing liquid. In turn, in FR3005742A1, a “TAC+product” (TAC: titre alkalimétrique complet) is disclosed, but accurate correction is not possible for various reasons. Indeed, in methods according to FR3005742A1, the LSI is not used, and work is done purely at intervals. Moreover, alkalinity measurement according to FR3005742A1 is done via observation of CO₂ concentration, which is complex and/or inaccurate.

Increasing alkalinity may be related to one or more of bicarbonates (HCO₃⁻) and carbonates (CO₃²⁻), hydroxides (OH⁻), sulphides (S₂⁻), silicates (SiO₄⁴⁻), phosphates (PO₄³⁻) and borates (BO₃³⁻). In embodiments, the alkalinity-increasing liquid is related to bicarbonates (HCO₃⁻) and/or carbonates (CO₃²⁻), preferably to bicarbonates.

According to further aspects of the invention, which are non-limiting, the invention relates to the following clauses 1-17.

Clause 1. Method for correcting alkalinity of water (7), preferably water from a swimming pool, comprising the steps:
automatically pumping a sample of said water (7) and a measuring liquid (1) comprising a known dilution of an acid corresponding to a known acidity, according to a known mixing ratio, to obtain a ready-to-measure solution;
measuring an acidity of said ready-to-measure solution by means of a pH sensor (8);
measuring, based on said acidity, said alkalinity of the water (7);
performing, based on said measurement, said correction with regard to the alkalinity of the water (7), by an automated and dosed addition of an alkalinity-increasing liquid (4) by means of an alka-plus pump (40).

Clause 2. Method according to clause 1, wherein said alkalinity-increasing liquid (4) comprises carbonate, CO₃²⁻, and/or bicarbonate, HCO₃⁻.

Clause 3. Method according to clause 2, wherein said alkalinity-increasing liquid (4) comprises said carbonate or bicarbonate at a concentration higher than 0.2 M.

Clause 4. Method according to clause 3, wherein said alkalinity-increasing liquid (4) comprises potassium, preferably potassium derived from dissolving potassium carbonate, K₂CO₃, and/or potassium bicarbonate, KHCO₃, in water, for obtaining the alkalinity-increasing liquid.

Clause 5. Method according to clause 3, wherein said alkalinity-increasing liquid (4) comprises sodium, preferably sodium derived from dissolving sodium carbonate, Na₂CO₃, and/or sodium bicarbonate, NaHCO₃, in water, for obtaining the alkalinity-increasing liquid.

Clause 6. Method according to clauses 1-5, wherein said automated pumping of respective samples of water (7) and of measuring liquid (1) and said measuring of acidity of said ready-to-measure solution and alkalinity of the water (7) is repeated periodically, and wherein said correction is performed by adding alkalinity-increasing liquid (4) when a difference between a target value for alkalinity and the measured alkalinity exceeds a certain threshold.

Clause 7. Method according to clause 6, wherein said dosage for correcting alkalinity of the water (7) with the addition of alkalinity-increasing liquid (4) corresponds to a corrected value not exceeding said target value, preferably lower than said target value.

Clause 8. Method according to clauses 1-7, wherein said measurement of said acidity is done by means of said pH sensor (8) on the basis of a bijective relationship between acidity of the ready-to-measure solution and alkalinity of the water (7) which is valid in a working area having said known acidity and said measurement of acidity.

Clause 9. Method according to clauses 1-8, wherein said diluted acid belonging to said measuring liquid (1) relates to dicarboxylic acid, preferably adipic acid, C₆H₁₀O₄.

Clause 10. Method according to clause 9, wherein said dicarboxylic acid is adipic acid, and wherein said dilution is preferably situated between 0.01 and 10 g/l, more preferably between 0.1 and 1 g/l, most preferably is 0.2 or 0.3 or 0.4 or 0.5 or 0.6 or 0.7 or 0.8 g/l.

Clause 11. Method according to clauses 1-10, wherein said known mixing ratio is between 1:10 and 10:1, preferably between 1:5 and 5:1, more preferably between 1:4 and 4:1 or between 1:3 and 3:1 or between 1:2 and 2:1, most preferably between 2:3 and 3:2 or between 3:4 and 4:3.

Clause 12. Method according to clauses 8-11, further comprising the step of:
measuring the temperature of said ready-to-measure solution by means of a temperature sensor;
wherein said measurement of acidity of said ready-to-measure solution is converted based on said temperature measurement for obtaining an equivalent pH, wherein said bijective relationship between acidity and alkalinity is applied to said equivalent pH for said measurement of alkalinity.

Clause 13. Method according to clauses 1-12, further comprising the step of:
switching between a first state (101) and a second state (301, 701), wherein said first state (101) corresponds to measuring alkalinity of the water (7) by means of said pH sensor (8); and wherein said second state (301, 701) corresponds to measuring acidity of the water (7) by means of the same pH sensor (8);
wherein said correction of alkalinity is done based on both said measurement of alkalinity of the water (7) and said measurement of acidity of the water (7).

Clause 14. Method according to clause 13, further comprising the step of:
performing, based on both said measurement of alkalinity of the water (7) and said measurement of acidity of the water (7), a correction with regard to the acidity of the water (7), by an automated and dosed addition of an acidity-reducing liquid (5) by means of a pH-min pump (50).

Clause 15. Method according to clauses 1-14, wherein said water (7) is the water of a swimming pool.

Clause 16. Use of a combination of a measuring liquid (1) comprising a known dilution of an acid corresponding to a known acidity comprising adipic acid and an alkalinity-increasing liquid (4) comprising a carbonate or bicarbonate for automatic and fluid-based correction of alkalinity of a swimming pool, preferably using a method according to clauses 1-15.

Clause 17. Kit for correcting alkalinity of a swimming pool, comprising:
a measuring liquid (1) comprising a known dilution of an acid corresponding to a known acidity comprising adipic acid; and
an alkalinity-increasing liquid (4) comprising a carbonate or bicarbonate;
wherein said alkalinity-increasing liquid (4) comprises carbonate, CO₃²⁻, and/or bicarbonate, HCO₃⁻; and
wherein said alkalinity-increasing liquid (4) comprises said carbonate or bicarbonate at a concentration higher than 0.2 M.

According to further aspects of the invention, which are non-limiting, the invention relates to the following items 1-17.

Item 1. Method for automatically regulating water quality (7), preferably water from a swimming pool, comprising the steps of:
actively measuring alkalinity of the water (7) with a first sensor, during a first state (101);
switching, from the first (101) to a second (301, 701) state;
measuring pH of the water (7) with the same first sensor, during the second state;
calculating, based on said measurement of alkalinity and pH, an index for water balance, preferably a Langelier Saturation Index, LSI;
determining whether the calculated index meets a predetermined criterion regarding said quality;
if said criterion is not met, generating an instruction for correction of said quality;
wherein said first sensor is a pH sensor (8);
wherein said first (101) and second (301, 701) states are at least partially non-overlapping in time, preferably non-overlapping in time;
wherein said active measurement relates to the automated and dosed addition of a measuring liquid to obtain a ready-to-measure solution.

Item 2. Method according to item 1, wherein said index for water balance is said LSI.

Item 3. Method according to items 1 or 2, wherein said criterion relates to a warning interval, wherein if the calculated index is not within said interval, an instruction is generated warning the operator.

Item 4. Method according to items 1-3, wherein said criterion relates to an interval for correction, wherein if the calculated index is not within said interval, an instruction for correction is generated, and wherein said method further comprises at least one of the following two steps:
if said instruction for correction includes increasing alkalinity, the automated and dosed addition of an alkalinity-increasing liquid (4) to the water by means of an alka-plus pump (40); and/or
if said instruction for correction includes lowering acidity, the automated and dosed addition of an acidity-reducing liquid (5) to the water by means of a pH-min pump (50).

Item 5. Method according to item 4, wherein said method includes both of the above two steps.

Item 6. Method according to items 1-5, wherein said measurement of said acidity by means of said pH sensor (8) is done based on a bijective relationship between acidity of the ready-to-measure solution and alkalinity of the water (7) which is valid in a working area having said known acidity and said measurement of acidity.

Item 7. Method according to items 1-6, wherein said active measuring relates to having a measuring liquid (1) mixed with a sample of the water (7) according to a known mixing ratio, the measuring liquid (1) comprising a known dilution of an acid corresponding to a known acidity, for obtaining the ready-to-measure solution.

Item 8. Method according to item 7, wherein said known mixing ratio is between 1:10 and 10:1, preferably between 1:5 and 5:1, more preferably between 1:4 and 4:1 or between 1:3 and 3:1 or between 1:2 and 2:1, most preferably between 2:3 and 3:2 or between 3:4 and 4:3.

Item 9. Method according to items 6-8, further comprising the step of:
measuring temperature of said ready-to-measure solution by means of a temperature sensor;
wherein said measurement of acidity of said ready-to-measure solution is converted based on said temperature measurement for obtaining an equivalent pH, wherein said bijective relationship between acidity and alkalinity is applied to said equivalent pH for said measurement of alkalinity.

Item 10. Method according to items 1-9, comprising the further step:
repeatedly switching between the first state (101) and the second state (301, 701);
in which said steps are repeatedly completed based on repeated measurement of alkalinity of the water (7), during the first state (101), and of acidity of the water (7), during the second state (301, 701).

Item 11. Method according to items 1-10, wherein said criterion is related to an interval for correction, wherein if the calculated index is not within said interval, an instruction for correction is generated, and wherein said method comprises the following step:
if said instruction for correction includes increasing salinity, the automated and dosed addition of a salinity-increasing liquid to the water (7) by means of a salt-plus pump.

Item 12. Method according to items 5-11, wherein said addition of alkalinity-increasing liquid (4) is done in priority to at least said addition of acidity-reducing liquid (5) in order to make the correction gradual.

Item 13. Method according to items 5-12, wherein said method comprises said following step:
if said instruction for correction includes increasing salinity, the automated and dosed addition of said salinity-increasing liquid to the water (7) by means of said salt-plus pump;
wherein said addition of salinity-increasing liquid is made in priority to at least said addition of acidity-reducing liquid (5) in order to make the correction gradual.

Item 14. Method according to items 1-13, wherein said water (7) is the water of a swimming pool.

Item 15. Device for automatically regulating water quality (7), comprising:
a first sensor;
means for performing an active measurement;
a control unit connected to said first sensor;
wherein said control unit is configured for:
measuring, by means of said first sensor, pH of the water (7), during an initial state;
actively measuring, by means of the same first sensor and said means for performing said active measurement, said alkalinity of the water (7), during a second state;
calculating, based on said measurement of pH and alkalinity, an index for water balance, preferably a Langelier Saturation Index, LSI;
determining whether the calculated index meets a predetermined criterion regarding said quality;
if said criterion is not met, generating an instruction for improving said quality;
wherein said first sensor is a pH sensor (8);
wherein said first and second states are at least partially non-overlapping in time, preferably non-overlapping in time;
wherein said means for performing the active measurement relate to the automated and dosed addition of a measuring liquid for obtaining a ready-to-measure solution.

Item 16. Swimming pool comprising the device according to item 15 comprising an inlet (2′) and outlet (2″) and a pool tub comprising said water (7) connected to said device by means of said inlet (2′) and outlet (2″).

Item 17. Swimming pool according to item 16, wherein said device regulates the quality of the water by means of a ready-to-measure solution and wherein said swimming pool comprises a further pump (90) realising an incoming flow (700′) through said device for draining the ready-to-measure solution to the pool tub.

In what follows, the invention will be described using non-limiting examples illustrating the invention, which are not intended or should not be interpreted to limit the scope of the invention.

### EXAMPLES

### Example 1: Exemplary Embodiment

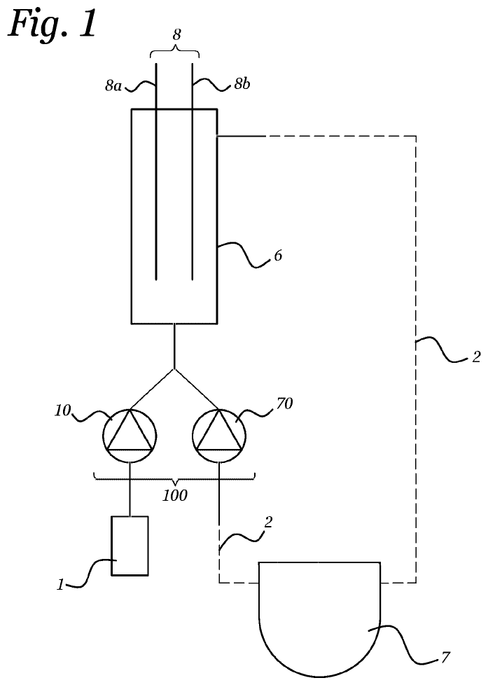

FIG. 1 shows a schematic exemplary embodiment of a device according to the invention, wherein the application is a swimming pool. In this embodiment, the mixing coil is omitted, as it is not essential for the operation of the device according to the invention.

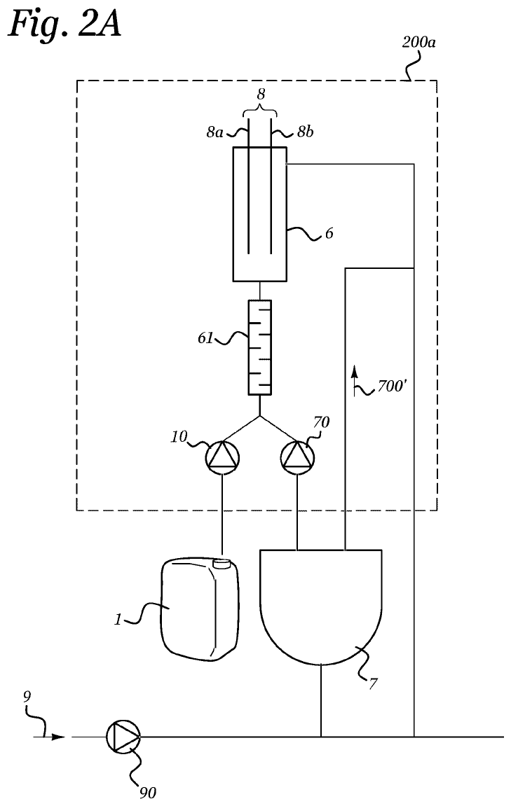

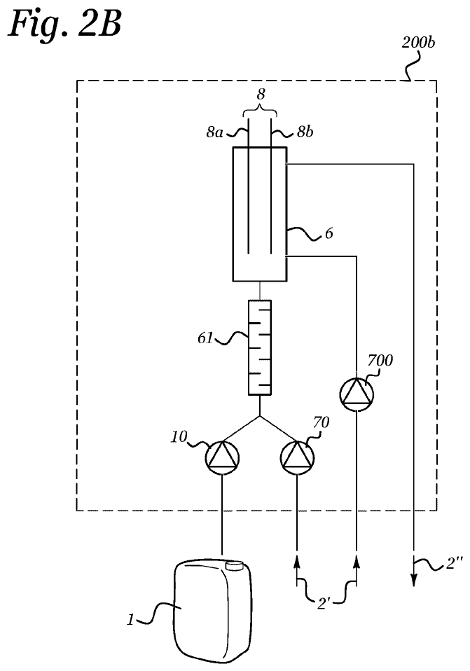

FIGS. 2A-2B show exemplary embodiments of devices according to the invention, wherein a mixing coil 61 is present. This may have the advantage of enhancing the mixing of measuring liquid 1 and sample of the water 7, resulting in an even more homogeneous ready-to-measure solution.

Thereby, FIG. 2A shows an example of the device 200a without incoming-flow pump, and

FIG. 2B shows an example of the device 200b with incoming-flow pump 700. Both examples 200a and 200b are largely equivalent in their operation, and the choice between the two is preferably determined by what is already present in the swimming pool. Specifically, in example 200a, the swimming pool installation, or, equivalently, the swimming pool, already includes a further pump 90 which realises a further flow 9, which in turn realises an incoming flow 700′. Thus, in example 200a, there is no need for an incoming-flow pump since flow 700′ is already strong enough to enable proper operation of the invention. It is unimportant here whether this further pump 90 was already present prior to installation of the device or not. Indeed, it is conceivable that pump 90 is provided at the same time as the installation of device 200a, or is replaced by a pump of sufficient power such that flow 700′ is sufficiently powerful. Thereby, pump 90 may realise various functions in the swimming pool, which may be related to further (temperature) distribution of the water and/or cleaning of the water and/or refreshing of the water, and the usefulness of pump 90 need not be limited to only maintaining incoming flow 700′.

FIG. 2B shows an exemplary embodiment of the device 200b with incoming-flow pump 700. In this example, in embodiment 200b, there is no need for an external pump to generate incoming flow 700′, as it is fully realised by incoming-flow pump 700. In still other embodiments (not shown), an external pump 90 is present which contributes to flow 700′, and the incoming-flow pump 700 only increases, decreases or flattens flow 700′, which may be more power-efficient and/or enable more accurate operation of the device.

A further difference between FIGS. 2A and 2B is that in FIG. 2A, the swimming pool with water 7 is shown, while in FIG. 2B, the inlet 2′ and outlet 2″ of the device are shown. This is not a difference between embodiments 200a and 200b but merely to illustrate a preferred embodiment with possibility of coupling and uncoupling of the devices, which is preferably possible with removable connections at the inlet and outlet. Removable connections may be advantageous during installation and/or repair and/or maintenance and/or calibration.

FIGS. 1, 2A-2B illustrate all embodiments of the device. Thereby, the device comprises:
a first pump 10 for automatically pumping a measuring liquid 1 comprising a known dilution of an acid corresponding to a known acidity;
a second pump 70 for automatically pumping a sample of the water 7;
a chamber 6 connected to said first 10 and second pump 70, for receiving a ready-to-measure solution comprising said sample and said measuring liquid 1 according to a known mixing ratio, the chamber 6 comprising a pH sensor 8 for measuring acidity; and
a control unit connected to said pH sensor 8.

The control unit (not shown) is configured to, based on a measurement of acidity of said ready-to-measure solution by means of said pH sensor 8 and based on a bijective relationship between acidity of the ready-to-measure solution and alkalinity of the water 7 which is valid in a working area having said known acidity and said measurement of acidity, measure said alkalinity. This bijective relationship can be determined, for example, from what is described in Example 4.

FIGS. 1, 2A-2B illustrate, in addition to the device, the system to which the device belongs. The system further comprises a source of measuring liquid 1, and a set of connections 2 for measuring alkalinity according to a closed circuit 2 with inlet 2′ connected to said second pump 70, and outlet 2″ connected to said chamber 6. This system in turn belongs to a swimming pool, comprising a pool tub comprising said water 7, and connected to said system by means of said inlet 2′ and outlet 2″.

FIG. 2B thereby further illustrates the possibility of using the device (either exemplary embodiment 200b, or exemplary embodiment 200a, or any other exemplary embodiment) for measurement of alkalinity of a limited amount of water, for example less than 10 l or less than 5 l or less than 1 l or less than 10 cl. In such examples, it may be advantageous to take a sample through said inlet 2′, but not to connect the outlet 2″ to the limited amount of water, and, instead, to drain the contents of the chamber, which may, for example, be filled with the ready-to-measure solution. In such embodiments, the closed circuit 2 is then not used, but only a flow from inlet to outlet. An advantage may be, for example, that the limited amount of water is then not disturbed by the addition of the ready-to-measure solution, and/or that it is avoided that the limited amount of water flows over the edges of the receptacle in which it is contained. This contrasts with embodiments where the amount of water is substantial, e.g. more than 1 l or more than 10 l, and where closed-circuit operation, with possible addition of measuring liquid to the amount of water, has only a limited or very limited influence on the composition of the water, due to the considerable dilution. Embodiments without a closed circuit have not been discussed further in the examples, but are equally part of the invention.

In exemplary embodiment 200b, the swimming pool also includes, in addition to the system that performs measurements of alkalinity with respect to a ready-to-measure solution, a further pump 90. The further pump realises, or contributes to, an incoming flow 700′ through said system. This incoming flow is useful for draining the ready-to-measure solution to the pool tub, and filling the chamber 6 with a sample of water 7 consisting only of the water itself, and no longer containing, or as little as possible, measuring liquid 1.

In this example, and also in examples 2 and 3, the first 10 and second 70 pumps are illustrated as separate pumps that, although belonging to a common pumping system 100, may be completely separated in space. In embodiments, the first and second pumps are located more than a metre away from each other, and are physical components completely separated from each other. In other example embodiments, not shown on the figures, the first 10 and second 70 pumps belong to one and the same pumping unit 100, and the pumping system 100 is thus an integrated system, with a common pump body. Thereby, the pumping system is, for example, a diaphragm pump or peristaltic pump. This allows a fixed transmission ratio to be achieved between the first and second pumps. In still other embodiments, the second pump 70 is actuated by means of a transmission of rotation of a shaft of the first pump 10 according to a fixed transmission ratio, or vice versa, i.e., where the first pump is actuated by the second one. This in turn corresponds to a pumping system 100 where there is partial integration between the first and second pumps. All these embodiments, either with partial mechanical integration or with full integration, have the advantage that a mechanically coupled volumetric ratio is realised through said fixed transmission ratio. These embodiments are not shown but are regarded as further examples which are equally described in this document.

In this example, and also in examples 2 and 3, the pH sensor 8 is illustrated as a set of a first 8a and second 8b electrode. However, the invention is not limited thereto, and any means suitable for pH measurement can be used, including holographic pH sensors with colorimetric operation, and including pH sensors with solid-state electrodes instead of conventional glass electrodes. These embodiments are not shown but are considered further examples which are equally described in this document.

In this example, the measuring liquid 1 is introduced from a reagent tank, and mixed with the water sample in a fixed ratio. Both streams are conveyed via the pumping device 100 to the chamber 6, with the mixing coil 61 in between for embodiments 200a and 200b. The chamber 6 functions as a detection cell, where pH as well as temperature are measured by the pH sensor 8. The pH of the mixture (=final pH, which is T-corrected via the temperature measurement) determines the alkalinity of the water.

To link the final pH to an alkalinity, a calibration is first performed, for example the calibration explained in example 4. To this end, in this example, the pH sensor 8 is first calibrated with at least two pH calibration points (e.g. 4.01 and 7.01 at 25° C.), and then calibration is carried out for alkalinity. To this end, the water tank is replaced with at least two calibration solutions. The device is activated, mixing each calibration solution with the measuring liquid, resulting in a final pH for each calibration solution. By reconnecting the water tank after calibration, the alkalinity of the water can be determined. The alkalinity of the sample can be measured continuously or per time interval.

The choice of the acid for the measuring liquid can be either a strong or a weak acid. The advantage of a weak acid may be that it also exhibits buffering capacity on its own within a certain interval. Because of this buffering capacity, the mixing of measuring liquid and sample and the subsequent measurement of the ready-to-measure solution are more predictable, and the final pH can potentially be determined more accurately. Adipic acid is particularly advantageous because of its non-toxic properties and ready availability.

The amount of the acid can be controlled by changing the mixing ratio and/or by changing the concentration. This can also help determine, and also limit, the net consumption of measuring liquid. The amount of acid can thereby be adjusted to the amount of alkalinity of the water. Thus, specific reagents can be made that can determine the alkalinity within a certain range, with e.g. a first solution for the range from 0 to 200 mg/l CaCO₃, and a second solution for the range from 200 to 400 mg/l.

After calibration of the device, the alkalinity of a sample can be determined (either with an internal, incoming-flow pump 700 or with an external pump 90). The alkalinity measurement can be analysed by time interval.

### Example 2: Exemplary Embodiments with Switching

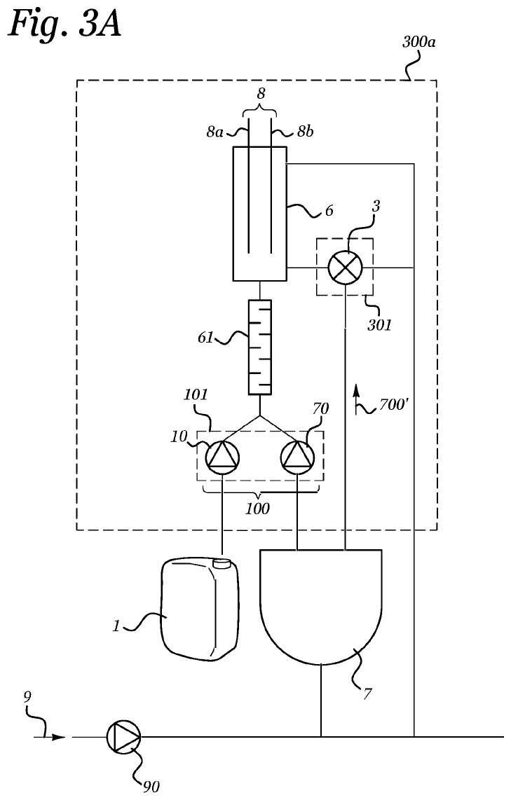

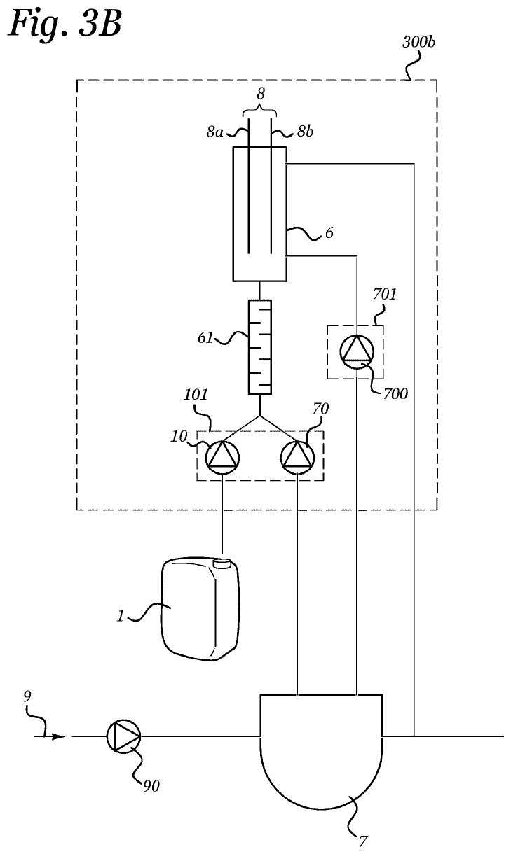

FIGS. 3A-3B show exemplary embodiments of devices according to the invention switching between a first and second state. Thereby, FIG. 3A shows an exemplary embodiment with valve, and FIG. 2B shows an exemplary embodiment with incoming-flow pump 700. The rationale for variants 300a and 300b is the same as for variants 200a and 200b in example 1.

In this example, alternating measurement of alkalinity and pH is possible, by alternating between first state 101 and second state 301, 701. In embodiment 300a, this relates to a valve 3, or a 3-way valve, which alternates between the first 10 and second 70 pump. In embodiment 300b, this relates to alternating between the pumping device 100 and the incoming-flow pump 700.

This relates to means for switching between a first state 101 and a second state 301, 701, wherein said first state 101 corresponds to measuring alkalinity of the water 7 by means of said pH sensor 8; and wherein said second state 301, 701 corresponds to measuring acidity of the water 7 by means of the same pH sensor 8.

For device 300a, said switching means are related to valve 3, preferably a three-way valve; wherein said first state 101 is associated with actuation of said first pump 10 and second pump 70 and with maintaining, by means of said valve 3, an incoming flow 700′ of water 7 without passage through the chamber 6, for said measurement of alkalinity by means of the pH sensor; and wherein said second state 301 is associated with non-actuation of said first pump 10 and second pump 70 and with guiding, by means of said valve 3, the incoming flow 700′ of water 7 through the chamber 6, for said measurement of acidity by means of the pH sensor.

For device 300b, said means of switching are related to an incoming-flow pump 700;

wherein said first state 101 is associated with actuation of said first pump 10 and second pump 70 and with non-actuation of the incoming-flow pump 700, for said measurement of alkalinity by means of the pH sensor; and wherein said second state 701 is associated with non-actuation of said first pump 10 and second pump 70 and with actuation of the incoming-flow pump 700 for guiding the incoming flow 700′ of water 7 through the chamber 6, for said measurement of acidity by means of the pH sensor.

### Example 3: Exemplary Embodiment with Alka-Plus Pump

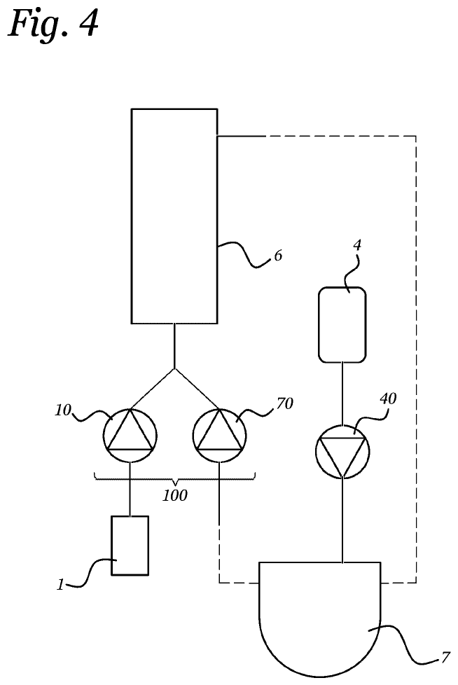

FIG. 4 shows a schematic exemplary embodiment of a device according to the invention with an alka-plus pump 40 with alkalinity-increasing liquid 4 coupled thereto.

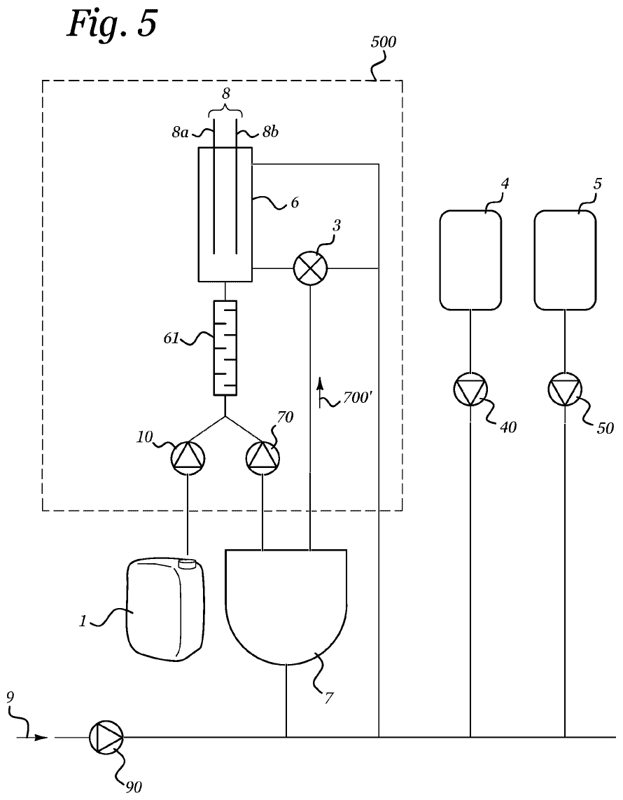

FIG. 5 shows an exemplary embodiment of a device 500 according to the invention with both an alka-plus pump 40 with alkalinity-increasing liquid 4 and a pH-min pump 50 with acidity-reducing liquid 5 coupled thereto.

With a device 500 and corresponding system, it is possible to alternately measure pH and alkalinity, and additionally administer pH and alka+. For example, it is possible to measure only pH during start-up, thus, to start in the second state. Also at times when the further pump 90 is on idle or still in start-up, it can be advantageous to measure pH rather than alkalinity. This may involve, for example, a 10-minute period during which pH is measured. Based on this measurement, preferably, no adjustments are made yet, because alkalinity should be measured first and adjusted as a priority, i.e. prior to any pH adjustment. Initially, alkalinity shouldn't be measured or adjusted yet because first, there should be sufficient circulation in the swimming pool. After the first ten minutes, the system preferably switches to the first state, where measurement of alkalinity is done next. Thereafter, the system cyclically switches between first and second state, whether or not according to a fixed schedule, alternately measuring alkalinity and pH and whether or not adjusting them. The time interval at which switching between pH and alkalinity occurs, is configurable.

### Example 4: Exemplary Embodiment Calculation

This example relates to the measurement of alkalinity according to the invention, wherein measurement is based on an “active measurement”. In this example, this relates to a measurement in which a measuring liquid (active) is added to a sample of the water to obtain a ready-to-measure solution. pH is then measured for the ready-to-measure solution, after which alkalinity is derived based on a bijective relationship between acidity of the ready-to-measure solution and alkalinity of the water. Example 4 may thereby be advantageously used for the alkalinity measurement of example 1 and/or 2 and/or 3.

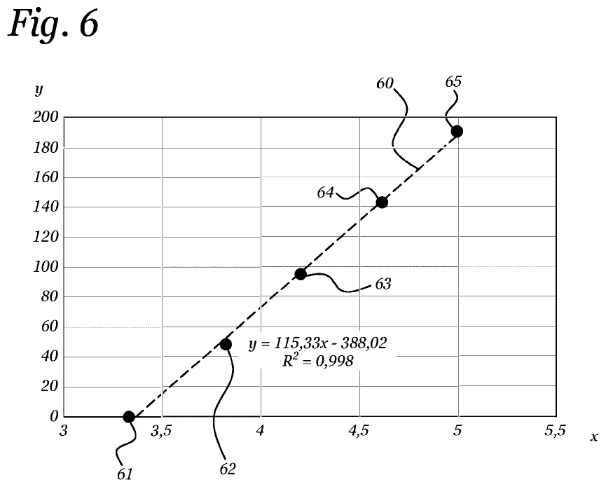

FIG. 6 shows an example of a bijective relationship according to the invention, wherein alkalinity of the water (y-axis, expressed in ppm) is plotted as a function of acidity of the ready-to-measure solution (x-axis, pH).

In this example, the measurement of alkalinity is related to the following steps:
calibrating the pH sensor 8 with at least two pH calibration solutions corresponding to a pH-related bijective calibration curve establishing a relationship between pH and a value measured by said pH sensor, in this example the voltage between a pair of electrodes 8a, 8b belonging to the pH sensor 8;
calibration with regard to alkalinity with at least two alkalinity-calibration solutions corresponding to the abovementioned bijective relationship between acidity of the ready-to-measure solution and alkalinity of the water 7;
having a measuring liquid 1 mixed with a sample of the water 7 according to a known mixing ratio, the measuring liquid 1 comprising a known dilution of an acid corresponding to a known acidity, to obtain a ready-to-measure solution;
performing, with respect to said ready-to-measure solution, a measurement of acidity by means of a pH sensor 8;
measuring said alkalinity of the water 7, based on said measurement of acidity and based on a bijective relationship between acidity and alkalinity which is valid in a working area having said known acidity and said measured acidity.

This example involves swimming pool water, where the working area has alkalinity levels from 0 to 200 mg/l.

The type of diluted acid for the measuring liquid is adipic acid with a concentration of 0.4 g/l. The mixing ratio is 1:1. Thus, the ready-to-measure solution consists of 1 part of water and 1 part of measuring liquid.

To link the final pH to an alkalinity, the pH sensor is first calibrated. This is done with at least two pH calibration points, in this example 4.01 and 7.01, at 25° C. The calibration results in a bijective calibration curve, in this example a calibration line (first-degree curve), which links the mV obtained to the real pH.

Subsequently, the calibration with regard to alkalinity is done. To this end, the water tank is replaced by at least two calibration solutions, which are fed via the inlet 2′ which is connected to the second pump 70. The device thereby pumps each calibration solution as a sample, mixing it with the same volume of the measuring liquid 1, which is pumped with the first pump 1. Together, both form the ready-to-measure solution, which is passed through the mixing coil 71, where they are optimally mixed. The ready-to-measure solution then enters chamber 6, where the pH of each calibration solution is measured. This results in a final pH per calibration solution. These measurements allow to determine the bijective relationship between acidity and alkalinity, in this example a line. In another example, five calibration solutions are used, corresponding to what is shown in FIG. 6. These are solutions with a concentration of 0, 47.5, 95, 143 and 190 ppm of NaHCO₃. This provides measurement points 61-65 on FIG. 6, with numerical values as shown. In another example, unrelated to FIG. 6, the same solutions with a concentration of 0, 47.5, 95, 143 and 190 ppm of NaHCO₃ are presented, and provide the following numerical values, not shown on FIG. 6:

| ppm NaHCO₃ | Final pH |
|---|---|
| 0 | 3.55 |
| 47.5 | 4.02 |
| 95 | 4.42 |
| 143 | 4.79 |
| 190 | 5.17 |

As illustrated by FIG. 6, an approximate mathematical equation 60 can be calculated from this type of measurements, representing the transformation from the measured final pH to the alkalinity of the aqueous stream, being the bijective relationship 60 between acidity and alkalinity. The calculation can be done by any approximation, in this example R², or any other method of regression, e.g. least square error or least absolute error.

By reconnecting the water tank with water 7 after this double determination, the alkalinity of the water 7 can be determined. The alkalinity of the sample can be measured continuously or per time interval. When a bijective relationship has been established, for example the bijective relationship 60 as shown in FIG. 60, and subsequently an unknown water sample is analysed according to the above procedure, and the pH of the ready-to-measure solution is 4.5, the alkalinity of the water sample is equal to 131 ppm.

### Example 5: Exemplary Embodiment Water Balance

This example concerns the regulation of water quality through the water balance. Here, water quality is regulated and controlled based on a combined input of two or more basic parameters, such as, for example, pH, alkalinity, temperature, hardness, conductivity, and weather forecast.

In the state of the art, automatic adjustment of a swimming pool, for example, is mostly provided on the basis of individual controls for each basic parameter. For example, the basic parameter pH is regulated on the basis of an analysis of pH. When pH is too high, adjustments are made to this single parameter. pH- is added to lower the pH to a defined fixed value.

In the case of regulation according to the exemplary embodiment according to the invention, regulation of water quality is based on an index, preferably based on the LSI.

At least two basic parameters (e.g. pH, alkalinity, hardness, conductivity, temperature, weather forecast) are thereby combined in this index, also called ‘water balance’. The index is (via the algorithm) a calculated numerical value based on at least two basic parameters. This value can take on all values from minus infinity to plus infinity.

In this example, preconditions have been defined from which adjustment of the water balance is required. Limits of the water balance are defined (maximum and minimum, with or without certain warning limits, i.e. an instruction for correction which is, for example, a warning to an operator).

Thereby, regulation is carried out based on input data of a combination of basic parameters—i.e. based on the water balance (instead of each time separately on one specific basic parameter) and wherein the basic parameters can be adjusted in combination or not. When the water balance value exceeds a warning limit (or maximum/minimum), adjustments can be made. The water balance is regulated based on the regulation of one or more basic parameters. The regulation is based on an algorithm implemented by the control unit, which ensures that the water balance remains within the predefined limits by adjusting one or more basic parameters. In exemplary embodiments, it is hereby possible to assign to each basic parameter a permissible range within which each basic parameter can vary, allowing further control and keeping the water balance between certain limits.

In exemplary embodiments, disinfection is measured in addition to water quality and, preferably, additionally adjusted.

In an exemplary embodiment, two or more parameters (e.g. pH, alkalinity, hardness, conductivity, temperature, weather forecast . . . ) are translated into the index via a formula. In embodiments, this relates to one or both of the following definitions for water balance:

LSI index: (pH)+(−0.00014554*(T in ° C.)*(T in ° C.)+0.02808929*(T in ° C.)+0.00408167)+ (0.43475046*In (Hardness calcium in ppm)−0.39514691)+ (0.43475046*In (Total alkalinity in ppm)+0.00485309)−(−0.0000000100*(EC in ppm)*(EC in ppm)+0.000122*(EC in ppm)−12.08) iW index: 166.6666*[(pH)+(−0.00014554*(T in ° C.)*(T in ° C.)+0.02808929*(T in ° C.)+0.00408167)+ (0.43475046*In (Hardness calcium in ppm)−0.39514691)+ (0.43475046*In (Total alkalinity in ppm)+0.00485309)−(−0.0000000100*(EC in ppm)*(EC in ppm)+0.000122*(EC in ppm)−12.08)]+100

In exemplary embodiments, limits for water balance are also set.
LSI
   Maximum: 0.3 (Scale forming)
   Warning limit high: 0.15
   Ideal: 0
   Warning limit low: −0.15
   Minimum: −0.3 (corrosive)
°iW
   Maximum: 150 (Scale forming)
   Warning limit high: 125
   Ideal: 100
   Warning limit low: 75
   Minimum: 50 (corrosive)

In embodiments where the instruction for correction includes a regulation, adjustments are also made. In one example, temperature is decreased (e.g. winter and no further warming of the water) and the water balance drops below the critical value. The table below shows an example where temperature causes a critical drop in the water balance, while the other basic parameters remain constant.

| | | Ppm | | mg/l CaCO3 | ppm | ° C. |
|---|---|---|---|---|---|---|
| LSI | water balance | ALKA | pH | Ca hardness | EC | T |
| 0.005 | 101 | 110 | 7.3 | 375 | 1503 | 30 |
| −0.440 | 27 | 110 | 7.3 | 375 | 1503 | 10 |

The regulation algorithm ensures that the water balance returns within acceptable limits. In this example, the regulation prioritises adjustment of alkalinity, with alka+ (being alkalinity-increasing liquid), with a variation of +10. This adjustment will also change the pH to, say, 7.35. The pH here is still within its limits (e.g. 7.2 to 7.4) so it will not be adjusted with pH- (or acidity-reducing liquid). Then, the algorithm can choose e.g. to first increase the conductivity by adding dissolved salt with a variation of 250. This combination led to an improvement in the water balance, bringing it back within acceptable limits.

| | | ppm | | mg/l CaCO3 | ppm | ° C. |
|---|---|---|---|---|---|---|
| LSI | water balance | ALKA | pH | Ca hardness | EC | T |
| −0.318 | 47 | 110 | 7.3 | 375 | 1503 | 15 |
| −0.252 | 58 | 120 | 7.35 | 375 | 1750 | 15 |

In this way, water quality is regulated based on combined basic parameters.

(END OF EXAMPLE 5)

It will be clear that the present invention is not limited to the embodiments described above and that some modifications or changes can be added to the described examples without revaluing the added claims (or clauses or items). For example, the present invention was described with respect to water from a swimming pool, but it may also be any other volume of water of which alkalinity and/or acidity and/or water balance and/or overall quality needs to be monitored. The invention, in its various aspects, is particularly appropriate for all applications where it is important that the water is non-toxic and/or should be suitable for human consumption.

1-61. (canceled)

62. Device for measuring alkalinity of water, preferably water from a swimming pool, comprising:
a first pump for automatically pumping a measuring liquid comprising a known dilution of an acid corresponding to a known acidity;
a second pump for automatically pumping a sample of the water;
a chamber connected to said first and second pump, for receiving a ready-to-measure solution comprising said sample and said measuring liquid according to a known mixing ratio, the chamber comprising a pH sensor for measuring acidity;
a control unit connected to said pH sensor;
wherein said control unit is configured to, based on a measurement of acidity of said ready-to-measure solution by means of said pH sensor and based on a bijective relationship between acidity of the ready-to-measure solution and alkalinity of the water which is valid in a working area having said known acidity and said measurement of acidity, measure said alkalinity.

63. Device of claim 62, wherein said known mixing ratio is related to a mechanically coupled volumetric ratio between the flow rate of the first pump and the flow rate of the second pump.

64. Device of claim 63, wherein
said first and second pump belong to one and the same pumping device, preferably a diaphragm pump or peristaltic pump, wherein said mechanically coupled volumetric ratio is realised by means of a common pump body,
or
said second pump is actuated by means of a transmission of rotation of a shaft of the first pump according to a fixed transmission ratio, or vice versa, wherein said mechanically coupled volumetric ratio is realised by means of said fixed transmission ratio, wherein said transmission of rotation is related to:
pulleys, said transmission ratio being related to a ratio of respective diameters of said pulleys; and/or
gears, said transmission ratio being related to a ratio of respective number of teeth of said gears.

65. Device of claim 62, wherein
said known mixing ratio relates to pumping a known volume of said measuring liquid, with said first pump, and/or a known volume of a sample of said water, with said second pump
and/or
said pH sensor is related to an electrode pair comprising a first and second electrode.

66. Device of claim 62, further comprising:
an outlet connected to said chamber, configured to drain the ready-to-measure solution to the water according to a closed circuit;
and/or
a mixing coil provided between said first pump and second pump and said chamber for mixing said measuring liquid and said sample of water to said ready-to-measure solution.
and/or
a temperature sensor for measuring temperature of said ready-to-measure solution;
wherein said control unit is further configured to measure the temperature of said ready-to-measure solution by means of the temperature sensor and to convert, based thereupon, said measurement of acidity for obtaining an equivalent pH, wherein said bijective relationship between acidity and alkalinity is applied to said equivalent pH for said measurement of alkalinity.

67. Device of claim 62, further comprising means for switching between a first state and a second state, wherein said first state corresponds to measuring alkalinity of the water by means of said pH sensor; and wherein said second state corresponds to measuring acidity of the water by means of the same pH sensor.

68. Device of claim 67, wherein said means of switching relate to:
a valve, preferably a three-way valve;
wherein said first state is associated with actuation of said first pump and second pump and with maintaining, by means of said valve, an incoming flow of water without passage through the chamber, for said measurement of alkalinity by means of the pH sensor; and
wherein said second state is associated with non-actuation of said first pump and second pump and with guiding, by means of said valve, the incoming flow of water through the chamber, for said measurement of acidity by means of the pH sensor;
or
an incoming-flow pump;
wherein said first state is associated with actuation of said first pump and second pump and with non-actuation of the incoming-flow pump, for said measurement of alkalinity by means of the pH sensor; and
wherein said second state is associated with non-actuation of said first pump and second pump and with actuation of the incoming-flow pump for guiding the incoming flow of water through the chamber, for said measurement of acidity by means of the pH sensor.

69. Device of claim 62, further comprising
an alka-plus pump for automatically adding, based on said measurement of alkalinity of the water, an alkalinity-increasing liquid to the water.

70. Device of claim 62, further comprising
a pH-min pump for automatically adding, based on one of the said measurements of acidity, an acidity-reducing liquid to the water, wherein said measurement of acidity relates to:
said measurement of acidity of said ready-to-measure solution; and/or
if available, said measurement of acidity of the water.

71. Device of claim 62, further comprising
a salt-plus pump for automatically adding, based on said measurement of alkalinity and/or based on one of the said measurements of acidity, a salinity-increasing liquid to the water.

72. Device of claim 67, comprising said alka-plus pump and said pH-min pump;
wherein said control unit is further configured to, based on repeatedly performing said measurement of alkalinity of the water and said acidity of the water, perform a correction with respect to the water, wherein the correction includes both the possibility of adding said alkalinity-increasing liquid by means of said alka-plus pump and the possibility of adding said acidity-reducing liquid by means of said pH-min pump and, preferably, further includes the possibility of adding said salinity-increasing liquid by means of said salt-plus pump.

73. Device of claim 72, wherein said adding of alkalinity-increasing liquid occurs in priority to at least said adding of acidity-reducing liquid so as to make the correction gradual.

74. Device of claim 72, wherein said correction includes the possibility of adding said salinity-increasing liquid by means of said salt-plus pump, and wherein said adding of salinity-increasing liquid occurs in priority to at least said adding of acidity-reducing liquid so as to make the correction gradual.

75. System comprising a device of claim 62, a source of measuring liquid and a set of connections for measuring alkalinity according to a closed circuit with inlet connected to said second pump, and outlet connected to said chamber.

76. Swimming pool comprising said system of claim 75 comprising said inlet and outlet and a pool tub comprising said water connected to said system by said inlet and outlet.

77. Swimming pool of claim 76, wherein said system performs measurements of alkalinity with respect to a ready-to-measure solution and wherein said swimming pool comprises a further pump realising an incoming flow through said system for draining the ready-to-measure solution to the pool tub.

78. Method for measuring alkalinity of water, preferably water from a swimming pool, comprising the steps of:
having a measuring liquid mixed with a sample of the water according to a known mixing ratio, the measuring liquid comprising a known dilution of an acid corresponding to a known acidity, to obtain a ready-to-measure solution;
performing, with reference to said ready-to-measure solution, a measurement of acidity by means of a pH sensor;
measuring said alkalinity of the water, based on said measurement of acidity and based on a bijective relationship between acidity and alkalinity which is valid in a working area having said known acidity and said measured acidity.

79. Method of claim 78, wherein said mixing is done by means of a first pump for said measuring liquid and a second pump for said sample, wherein the first and second pumps are mechanically coupled according to a volumetric ratio between the flow rate of the first pump and the flow rate of the second pump.

80. Method of claim 78, wherein said steps are preceded by the steps of:
calibrating the pH sensor with at least two pH calibration solutions corresponding to a pH-related bijective calibration curve that relates pH to a value measured by said pH sensor, preferably the voltage between a pair of electrodes belonging to the pH sensor;
calibration with regard to alkalinity with at least two alkalinity calibration solutions corresponding to the abovementioned bijective relationship between acidity of the ready-to-measure solution and alkalinity of the water.

81. Use of a pumping device for measuring alkalinity by pumping a sample of water and a measuring liquid at a known mixing ratio by means of devices, belonging to the pumping device, for achieving a mechanically coupled volumetric ratio.
